(19) United States
(12) Patent Application Publication
(10) Pub. No.: US 2004/0162499 A1
(43) Pub. Date: Aug. 19, 2004

(54) Abnormal respiration detecting system and method for detecting the same

(76) Inventors: Fumiya Nagai, Anjo-city (JP); Shinji Nanba, Kariya-city (JP); Toshiaki Shiomi, Nagoya-city (JP)

Correspondence Address: POSZ & BETHARDS, PLC, 11250 ROGER BACON DRIVE, SUITE 10, RESTON, VA, 20190, US

(21) Appl. No.: 10/678,130

(22) Filed: Oct. 6, 2003

(30) Foreign Application Priority Data
Oct. 4, 2002 (JP) 2002-292695

(51) Int. Cl. A61B 5/08

(52) U.S. Cl. 600/529

(57) ABSTRACT

An abnormal respiration detecting system includes a sensing device for sensing a sphygmographic signal and a control device for determining a respiratory condition. The control device calculates a pulse rate, an amplitude of a pulse wave, a ratio between the pulse rate and the amplitude, and a respiration curve. The sensing device also senses a signal indicative of an oxygen saturation level in blood. The control device detects abnormal respiration based on a variation in the pulse rate and the ratio, a respiration rate, apnea, an apneic period that are calculated from the respiration curve, and the oxygen saturation level. Each abnormal respiration is rated and an overall abnormal respiration is rated. A warning is produced when the overall rating is equal to or higher than a predetermined level.

### CROSS REFERENCE TO RELATED APPLICATION

This application is based on and incorporates herein by reference Japanese Patent Application No. 2002-292695 filed on Oct. 4, 2002.

### FIELD OF THE INVENTION

The present invention relates to an abnormal respiration detecting system, a program for the system, recording media, a sensing device, and a method for detecting abnormal respiration.

### BACKGROUND OF THE INVENTION

A respiratory monitor for monitoring respiratory conditions of a patient during sleep is proposed in JP-A-2001-340309. In this monitor, pressure sensors are mounted to a bed. The respiratory conditions cannot be precisely monitored by this monitor since the sensors are not directly attached to the patient.

A method for determining respiratory conditions of a patient during sleep is proposed in U.S. Pat. No. 5,275,159 (JP-A-5-200031). This method is called a polysomnogram in which sensors are attached to the patient. In this method, a number of sensors (sensing electrodes) are hooked up to a person. Therefore, it is not convenient for applying this method to respiratory monitoring in places such as hospitals where many people have to be under monitoring. Moreover, it is not convenient for home uses.

Another method for detecting obstructive apnea based on sphygmographic signals detected by a pulse oximeter is proposed in U.S. Pat. No. 5,385,144 (JP-A-6-38965). In this method, obstructive apnea is detected based on variation in a base line of the sphygmographic signals. Therefore, abnormal respiratory conditions are not precisely detected.

### SUMMARY OF THE INVENTION

The first objective of the present invention is to provide an abnormal respiration detecting system. An abnormal respiration detecting system of the present invention includes a sensing means, a signal detecting means, a respiratory condition determining means, and an abnormal respiration detecting means.

The sensing means sense a signal indicative of physical sings of a person under the abnormal respiration detection. The signal detecting means detects a signal outputted from the sensing means. The respiratory condition determining means determines a respiratory condition based on the signal. The abnormal respiration detecting means detects abnormal respiration based on the determined respiratory condition.

For example, if the signal is a sphygmographic signal, the respiratory condition determining means calculates a pulse rate and an amplitude of a pulse wave from the sphygmographic signal. The abnormal respiration determination means determines abnormal respiration based on the calculated pulse rate and the amplitude.

When abnormal respiration occurs, the pulse rate and the amplitude of the sphygmographic signal show irregularity. Therefore, abnormal respiration can be detected based on the pulse rate and the amplitude from the sphygmographic signal. In comparison with the respiratory monitors and other methods that have been proposed, the system of the present invention provides higher accuracy in abnormal respiration detection. Furthermore, a configuration of the system is more simplified than that of a system using polysomnogram.

The second objective is to provide programs for executing the means of the abnormal respiration detecting system. The programs enable various functions for providing highly accurate abnormal respiration detection performed by the system.

The third objective is to provide a sensing device for sensing a signal indicative of physical sings of a person under the abnormal respiration detection. The device includes an optical pulse wave sensor and an optical pulse oximeter. The pulse wave sensor detects pulse waves using a blue or a green ray. The optical pulse oximeter measures oxygen saturation levels in blood using at least one of a red ray and a near-infrared ray.

The fourth objective is to provide a method for detecting abnormal respiration. A method of the present invention includes signal sensing, signal detecting, respiratory condition determining, and abnormal respiration detecting. A signal indicative of a physical sign is sensed by a sensing means, and a signal outputted from the sensing means is detected. A respiratory condition is determined based on the detected signal, and abnormal respiration is detected based on the determined respiratory condition. The method provides accurate abnormal respiration detection without requiring complex devices or systems.

### BRIEF DESCRIPTION OF THE DRAWINGS

The above and other objectives, features and advantages of the present invention will become more apparent from the following detailed description made with reference to the accompanying drawings. In the drawings:

FIG. 1 is an explanatory view of an abnormal respiration detecting system according to an embodiment of the present invention; a plan view

FIG. 2 is a waveform of a sphygmographic signal according to the embodiment;

FIG. 3 is a flowchart of an abnormal respiration detecting process according to the embodiment;

FIG. 4 is a waveform of a sphygmographic signal detected under an abnormal respiration condition according to the embodiment;

FIG. 5 is waveforms of a sphygmographic signal and envelope curves according to the embodiment; and

FIG. 6 is a waveform of a respiration signal according to the embodiment.

### DETAILED DESCRIPTION OF THE PREFERRED EMBODIMENT

The preferred embodiment of the present invention will be explained with reference to the accompanying drawings.

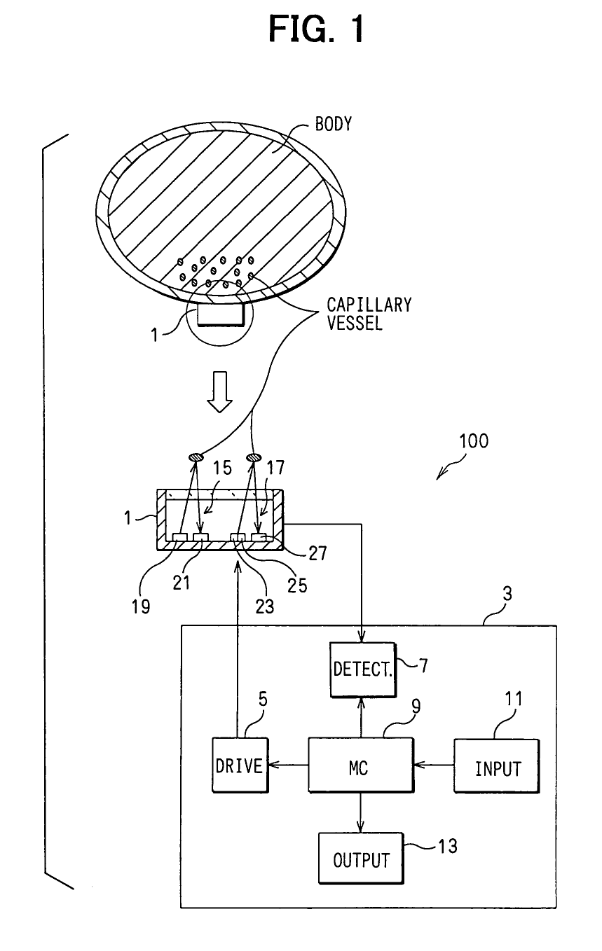

Referring to FIG. 1, an abnormal respiration detecting system 100 includes a sensing device 1 and a control device 3. The sensing device 1 is attached to a body of a person (a subject) under the abnormal respiration detection. It is preferable that the sensing device is attached to which a movement is less likely to occur, such as a wrist and a torso. The control device 3 includes a driving circuit 5, a signal detecting circuit 7, a data processing circuit 9 (microcomputer), an input circuit 11, and an output circuit 13.

The driving circuit 5 drives the sensing device 1. The detecting circuit 7 detects signals outputted from the sensing device 1. The data processing circuit 9 calculates pulse waves based on the signals converted from analog to digital and outputted from the detecting circuit 7, and calculates a pulse rate from the pulse waves. The input circuit 11 includes switches and sends an instruction signal to the data processing circuit 9. The output circuit 13 outputs data processed by the data processing circuit 9 in the form of display or sound (or voice).

The sensing device 1 is constructed of a pair of optical sensors, an optical pulse wave sensor 15 and a pulse oximeter 17. The pulse wave sensor 15 uses a blue or green ray for detecting pulse wave of an artery. The pulse oximeter 17 uses red and near-infrared rays for detecting an oxygen saturation level in the artery.

The pulse wave sensor 15 includes a light emitting device (LED) 19 and a photoreceptor device (PD) 21. The LED 19 is a blue light emitting device made of an Indium-Gallium-Nitrogen (InGaN) based material. The LED 19 is a wavelength converter that converts a wavelength by passing a beam of emitted light through fluorescent paint coated on an acrylic plate. The PD 21 is a photoreceptor device made of a Gallium-Arsenide-Phosphorus (GaAsP) based material.

The LED 19 has light emitting characteristics that show two peaks. The first and the second peaks correspond to a peak of an absorptive wavelength characteristic curve of hemoglobin and that of a sensitivity characteristic curve of the photoreceptor device, respectively. The first peak appears around 440 nm and the second peak appears around 550 nm. Therefore, the pulse wave sensor 15 has an SN ratio of 1/60, which is higher than a regular value. The SN ratio is a ratio of a signal component S that varies according to a variation in blood volume to the total amount of received light N.

The oximeter 17 is a known device constructed of a red LED (R-LED) 23, a near-infrared LED (IR-LED) 25, and a photoreceptor device (PD) 27. The PD 19 and the PD 27 are configured so that their light emitting timings differ from each other and light reception and signal processing of the PDs 19, 27 are separately performed. Light reception and signal processing of the R-LED 23 and the IR-LED 25 are performed by a time-sharing method that is commonly used.

An operation of the abnormal respiration detecting system 100 using the pulse wave sensor 1 will be discussed. A blue ray is emitted from the LED 19 driven by the driving circuit 5 toward an area in which the sensor 1 is placed. A part of the ray is absorbed by hemoglobin in capillary blood vessel, and other parts of the ray repeatedly scatter and some of them incident to the PD 11.

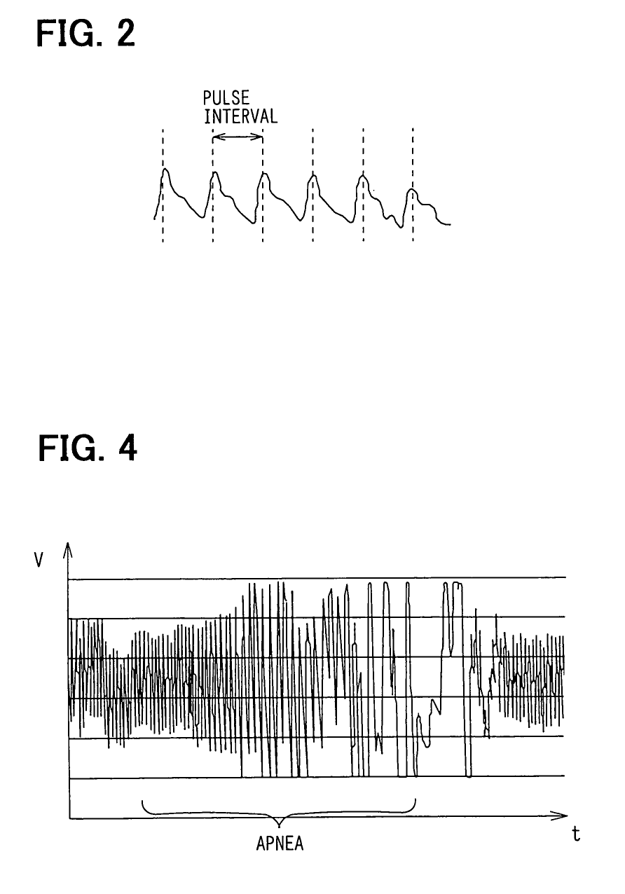

Since the amount of hemoglobin in the capillary blood vessel fluctuates according to blood pulsation (pulse), the amount of ray absorbed by hemoglobin fluctuates. The amount of light absorbed in the capillary blood vessel. As a result, the amount of light detected by the PD 21 varies, and the variation is inputted to the data processing device 9 in the form of voltage signals that indicate a sphygmographic waveform shown in FIG. 2. The waveform provides a blood flow volume.

The signal outputted from the pulse wave sensor 1 is amplified by the detecting circuit 7, noise-filtered by a band-pass filter circuit (not shown), converted to a digital signal, and inputted to the data processing circuit 9. The data processing circuit 9 calculates a pulse rate and pulse intervals from the inputted signal and detects abnormal respiration.

An operation of the detecting system 100 using the pulse oximeter 17 is basically the same as using the pulse wave sensor 1. A red ray or a near-infrared ray is emitted from the LED 23, 25 to a body of a person under the abnormal respiration detection at which the sensing device 1 is placed. The ray reflected off the body is received by the PD 27. A signal converted from the received ray varies according to a variation of the oxygen saturation level. Therefore, the oxygen saturation level is calculated by the data processing circuit 9 by processing the signal.

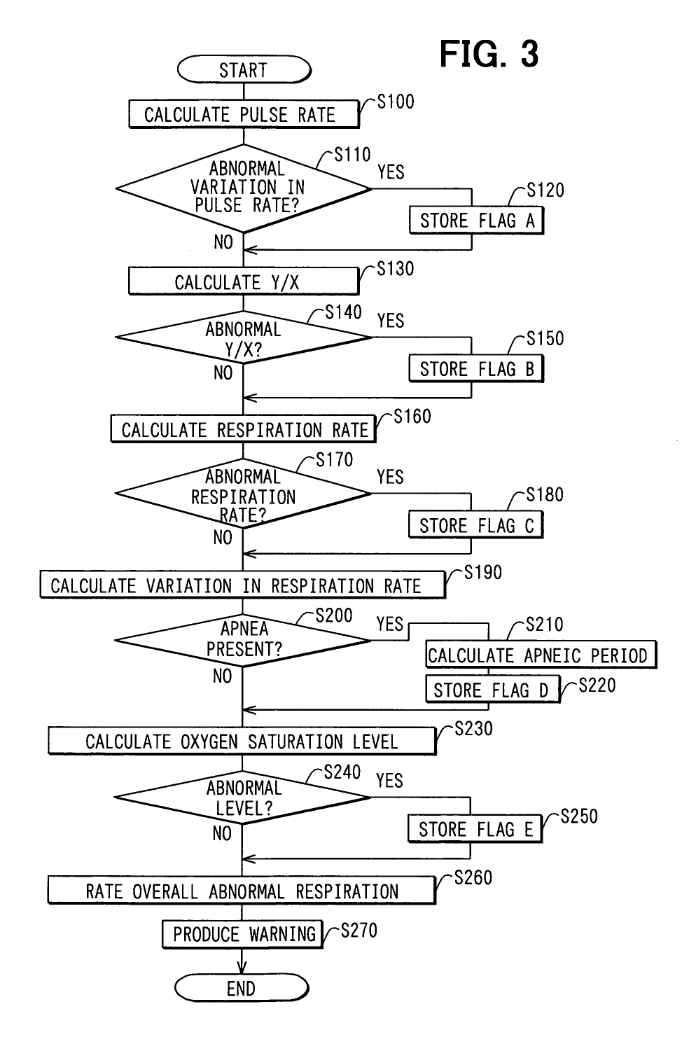

Referring to FIG. 3, a pulse rate is calculated (S100). A peak of each wave cycle in continuous sphygmographic signals corresponds to each pulse as shown in FIG. 2. Thus, the number of pulses per unit time, for instance per minute, is calculated from the number of peaks per unit time. It is determined whether a variation in the number of pulses is detected (S110). More specifically, it is determined whether any one of bradycardia, tachycardia, and arrhythmia is detected. Bradycardia, tachycardia, and arrhythmia are conditions that a pulse rate increases, decreases and varies, respectively.

When any irregular condition, such as shown in FIG. 4, is detected, an abnormal condition flag A, for instance 1, is stored as a result of abnormal condition detection A (S120). The abnormal condition detection A is performed for detecting an abnormal condition of the pulse rate. The flag A indicates seriousness of the abnormal pulse rate. The flag A and other abnormal condition flags B-E, which are set based on a result of respective abnormal condition detection B-E, are used for an overall abnormal respiration rating. The overall abnormal respiration rating is performed for determining whether a warning is necessary. The results of the detection A-E effect differently to the overall rating. Therefore, the flags A-E are differently weighted.

The ratio between amplitude of the pulse wave (Y) and the pulse rate (X) is calculated (S130). When respiration stops during sleep, bradycardia progresses and the amplitude of the pulse wave may increase to obtain necessary blood flow. This variation is detected by a variation in the pulse wave (Y) to the pulse rate (X) ratio (Y/X).

It is determined whether the ratio Y/X is abnormal by determining an increase in the ratio Y/X is within the normal range (S140). If the increase is larger than the normal range due to an increase in the amplitude Y and a decrease in the pulse rate X, it is determined that an abnormal condition has occurred.

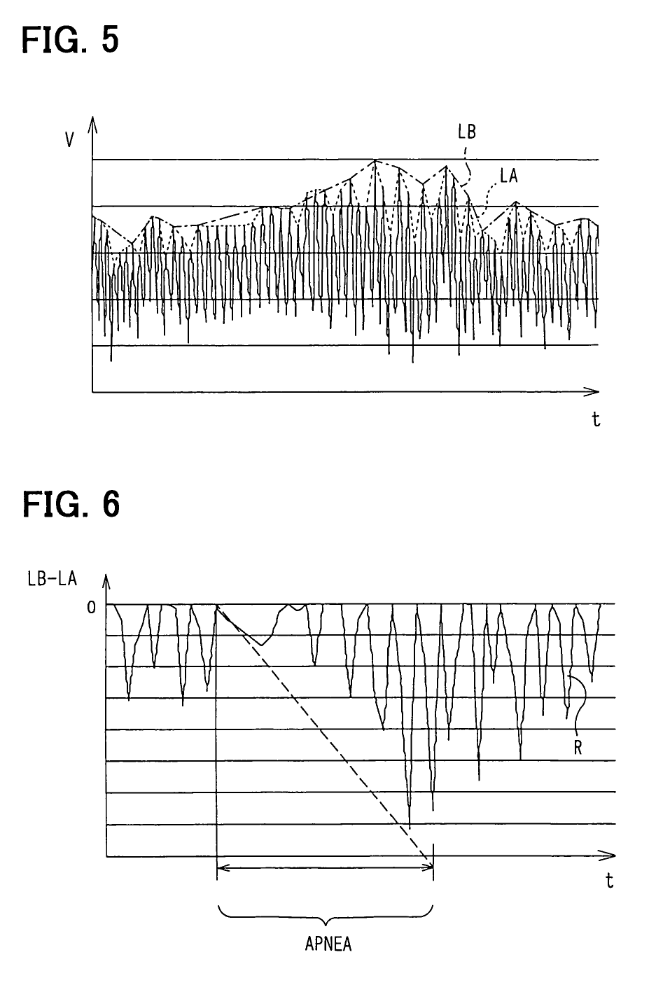

If it is determined that the ratio Y/X is abnormal, the abnormal condition flag B, for instance 3, is stored as a result of abnormal condition detection B (S150), and a respiration rate is calculated (S160). In the respiration rate calculation, an envelope curve LA is drawn by connecting peaks of pulse wave cycles and another envelope curve LB is drawn by connecting peaks of the envelope curve LA as shown in FIG. 5. A curve of the difference between the envelope curve LA and the other envelope curve LB (LB-LA) is regarded as a respiration curve R that express respiration conditions. Downward peaks represent inspiration.

It is determined whether the respiration rate is abnormal by judging the respiration rate is within a normal range. If the respiration rate is determined abnormal, the abnormal condition flag C, for instance 1, is stored as a result of abnormal condition detection C (S180). After the flag C is stored or if the respiration rate is normal, a variation in respiration signal is calculated (S190).

When the amplitude (LB-LA) of the respiration signal is increasing as shown by a dotted line in FIG. 6, the person is trying to enlarge the chest cavity to inhale air. In other words, the person is trying to increase a negative pressure in the chest cavity. This condition may occur when the person is in apnea. The variation in peaks of the respiration signal is calculated for detecting the apnea.

It is determined whether the apnea is present by judging the variation in peaks is within the normal range (S200). If the apnea is present, a period between increasing in peaks and decreasing in peaks (apneic period) is calculated (S210). The abnormal flag D having a value corresponding to the apneic period is stored as a result of abnormal condition detection D. After the flag D is stored or the apnea is not present at step S200, the oxygen saturation level in blood is calculated based on a value measured by the pulse oximeter 17 (S230).

It is determined whether the Oxygen saturation level is abnormal by judging the measured value is within a normal range (S240). If the Oxygen saturation level is abnormal, the abnormal condition flag E is stored as a result of abnormal condition detection E. After the flag E is stored or the oxygen saturation level is determined normal at step S240, the overall abnormal respiration rating is made based on the results of the abnormal condition detection A to E (S260).

The values of the abnormal condition flags A to E are summed and the overall abnormal respiration is rated. If the rating is equal to or higher than a predetermined level, it is determined that a warning is necessary. For instance, if the sum is equal to or more than 3, the abnormal respiration is serious and warning will be produced.

The abnormal respiration is reported in the form of display, sound or voice by driving the output circuit 13. In other words, a warning is produced according to the seriousness of abnormal respiration. Then, the process completes.

The pulse rate, the amplitude of pulse wave, and the oxygen saturation level are calculated using the pulse wave sensor 15 and the pulse oximeter 17 that are integrally included in the device 1. Therefore, the accuracy of the abnormal respiration detection improves in comparison with a system having only a respiration monitor or a pulse oximeter. Furthermore, the operation of the system is easier than that of the polysomnogram.

The overall abnormal respiration detecting is made based on the variation in pulse rate and respiration rate, apneic and apneic periods, and the variation in the oxygen saturation level in addition to the ratio Y/X. Furthermore, the seriousness of each condition is taken into consideration when the judgments are made. As a result, the abnormal respiration determinations are precisely made.

The present invention should not be limited to the embodiment previously discussed and shown in the figures, but may be implemented in various ways without departing from the spirit of the invention. For example, the abnormal respiration determination may be made based on an increase in occurrence of apnea per unit time more than predetermined times in addition to the conditions described above. In this case, the accuracy of the determination further improves.

Combinations of determination conditions may be used for an overall determination other than the amplitude of pulse wave and pulse rate. Electronic control devices, microcomputers, microchips, flexible disks, hard disks, and optical disks may be used for storage media for storing the programs for the system. The program may be up or downloaded via a communication line including internet.

Signals outputted from the sensing device may be inputted to a personal computer in the form of data or stored in storage media. Then, the data may be transmitted to a control device at a remote location via internet or other communication lines for diagnosis or examination. The data may be stored for later use. The respiration curve may be drawn by connecting points a predetermined value lower than the peaks. Various ways for drawing the respiration curves may be applied as long as they express the variation of the original waveform.

What is claimed is:

1. An abnormal respiration detecting system comprising:
a sensing means for sensing a signal indicative of a physical sign;
a signal detecting means for detecting a signal outputted from the sensing means;
a respiratory condition determining means for determining a respiratory condition based on the signal; and
an abnormal respiration detecting means for detecting abnormal respiration based on the determined respiratory condition.

2. The abnormal respiration detecting system according to claim 1, wherein:
the sensing means senses a sphygmographic signal; and
the respiratory condition determining means calculates a pulse rate and an amplitude of a pulse wave from the sphygmographic signal.

3. The abnormal respiration detecting system according to claim 2, wherein the respiratory condition determining means calculates a ratio between the pulse rate and the amplitude of the pulse wave.

4. The abnormal respiration detecting system according to claim 1, wherein:
the sensing means senses a sphygmographic signal;
the respiratory condition determining means calculates a respiratory curve from the sphygmographic signal and a respiration rate from the respiratory curve; and
the abnormal respiration detecting means detects the abnormal respiration based on a variation in the respiration rate.

5. The abnormal respiration detecting system according to claim 1, wherein:
the sensing means senses a sphygmographic signal;
the respiratory condition determining means calculates a respiratory curve from the sphygmographic signal and determines whether apnea is present based on the respiratory curve; and
the abnormal respiration detecting means detects the abnormal respiration based on the determination of apnea.

6. The abnormal respiration detecting system according to claim 1, wherein:
the sensing means senses a sphygmographic signal;
the respiratory condition determining means calculates a respiratory curve from the sphygmographic signal and determines whether apnea is present based on the respiratory curve;
the respiratory condition determining means determines an apneic period when apnea is present; and
the abnormal respiration detecting means detects abnormal respiration based on the apneic period.

7. The abnormal respiration detecting system according to claim 1, wherein:
the sensing means senses a sphygmographic signal;
the respiratory condition determining means calculates a respiratory curve from the sphygmographic signal and determines whether apnea is present based on the respiratory curve;
the abnormal respiration detecting means detects abnormal respiration based on a continuous variation of apnea when apnea is present.

8. The abnormal respiration detecting system according to claim 2, wherein the abnormal respiration detecting means detects abnormal respiration based on a variation in the pulse rate.

9. The abnormal respiration detecting system according to claim 1, wherein:
the sensing means senses a signal indicative of an oxygen saturation level in blood;
the respiratory condition determination means calculates an oxygen saturation level in blood from the signal;
the abnormal respiration detecting means detects abnormal respiration based on a variation in the oxygen saturation level in blood.

10. The abnormal respiration detecting system according to claim 1, further comprising:
an abnormal respiration rating means for rating the abnormal respiration based on seriousness of abnormal respiration;
a rating storing means for storing the rating of the abnormal respiration; and
an overall abnormal respiration rating means for rating an overall abnormal respiration to determine whether a warning is necessary;
a warning means for producing a warning when the overall abnormal respiration is determined that a warning is necessary, wherein
the respiratory condition determining means determines a plurality of respiratory conditions,
the abnormal respiration detecting means detects abnormal respiration based on each respiratory condition,
the abnormal respiration rating means rates each abnormal respiration, and
the overall abnormal respiration rating means rates an overall abnormal respiration based on the rating of each abnormal respiration.

11. The abnormal respiration detecting system according to claim 1, wherein:
the sensing means is an optical pulse wave sensor having one of blue and green light emitting devise;
the signal detecting means detects a sphygmographic signal inputted from the optical pulse wave sensor; and
the abnormal respiration detecting means detects abnormal respiration based on the sphygmographic signal.

12. The abnormal respiration detecting system according to claim 11, wherein:
the pulse wave sensor includes a light emitting device that emits light toward a subject of the abnormal respiration detection and a photoreceptor device that receives light emitted from the light emitting device and reflected off the subject; and
the light emitting device has emitting wavelength characteristics that shows two peaks, a first peak corresponding to a peak of an absorptive wavelength characteristic curve of hemoglobin and a second peak corresponding to a peak of a sensitivity characteristic curve of the photoreceptor device.

13. The abnormal respiration detecting system according to claim 11, wherein:
the first peak is about 440 nm; and
the second peak is about 550 nm.

14. The abnormal respiration detecting system according to claim 9, the sensing means is an optical pulse oximeter having at least one of a red light emitting device and a near-infrared light emitting device.

15. An abnormal respiration detecting program for executing at least one of means claimed in claim 1.

16. A storing media for storing the program claimed in claim 15 and data outputted during an execution of the program.

17. A sensing device comprising:
an optical pulse wave sensor having one of a blue light emitting device and a green light emitting device for detecting a pulse wave; and
an optical pulse oximeter having a red light emitting device.

18. The sensing device according to claim 17, wherein the pulse wave sensor including:
a light emitting device that emits light toward a subject of the abnormal respiration detection; and
a photoreceptor device that receives light emitted from the light emitting device and reflected off the subject, wherein
the light emitting device has emitting wavelength characteristics that shows two peaks, a first peak corresponding to a peak of an absorptive wavelength characteristic curve of hemoglobin and a second peak corresponding to a peak of a sensitivity characteristic curve of the photoreceptor device.

19. The sensing device according to claim 17, wherein:
the first peak is about 440 nm; and
the second peak is about 550 nm.

20. The sensing device according to claim 17, wherein the optical pulse wave sensor and the optical pulse oximeter are integrally constructed.

21. A method for detecting abnormal respiration comprising:
sensing a signal indicative of a physical sign by a sensing means;
detecting a signal outputted from the sensing means;
determining a respiratory condition based on the detected signal; and
detecting abnormal respiration based on the determined respiratory condition.

22. The method according to claim 21, wherein:
the signal outputted from the sensing means is a sphygmographic signal; and
the respiratory condition determining step further comprises calculating a pulse rate and an amplitude of the pulse wave from the sphygmographic signal.

23. The method according to claim 22, wherein the respiratory condition determining step further comprises calculating a ratio between the pulse rate and the amplitude.

24. The method according to claim 21, wherein:
the signal outputted from the sensing means is a sphygmographic signal;
the respiratory condition determining step further comprises calculating a respiratory curve from the sphygmographic signal and a respiration rate from the respiratory curve; and
the abnormal respiration is detected based on a variation in the respiration rate.

25. The method according to claim 21, wherein:
the signal outputted from the sensing means is a sphygmographic signal;
the respiratory condition determining step further comprises calculating a respiratory curve from the sphygmographic signal; and
the determining step further comprises determining whether apnea is present based on the respiratory curve.

26. The method according to claim 25, wherein:
the signal outputted from the sensing means is a sphygmographic signal;
the respiratory condition determining step further comprises calculating a respiratory curve from the sphygmographic signal; and
the determining step further comprises calculating an apneic period in which the apnea continues from the respiratory curve.

27. The method according to claim 25, wherein:
the signal outputted from the sensing means is a sphygmographic signal;
the respiratory condition determining step further comprises calculating a respiratory curve from the sphygmographic signal; and
the abnormal respiration is detected based on a continuous variation of apnea.

28. The method according to claim 21, wherein: the signal outputted from the sensing means is a sphygmographic signal;
the respiratory condition determining step further comprises calculating a pulse rate from the sphygmographic signal; and
the abnormal respiration is detected based on a variation in pulse rate.

29. The method according to claim 21, wherein:
the signal outputted from the sensing means is a signal indicative of an oxygen saturation level in blood;
the respiratory condition determining step further comprises calculating an oxygen saturation level in blood from the signal;
the abnormal respiration is detected based on a variation in the oxygen saturation level in blood.

30. The method according to claim 21, further comprising:
rating the abnormal respiration based on seriousness of abnormal respiration;
storing the rating of the abnormal respiration; and
rating an overall abnormal respiration to determine whether a warning is necessary, wherein
the respiratory condition determining step determines a plurality of respiratory conditions,
the abnormal respiration detecting step detects abnormal respiration based on each respiratory condition,
the abnormal respiration rating step rates each abnormal respiration, and
the overall abnormal respiration rating step rates an overall abnormal respiration based on the rating of each abnormal respiration.
